(19) United States
(12) Patent Application Publication
(10) Pub. No.: US 2014/0080885 A1
(43) Pub. Date: Mar. 20, 2014

(54) METHODS OF TREATING TRAUMATIC BRAIN INJURY

(71) Applicants: Keith R. Pennypacker, Wesley Chapel, FL (US); Shyam S. Mohapatra, Tampa, FL (US)

(72) Inventors: Keith R. Pennypacker, Wesley Chapel, FL (US); Shyam S. Mohapatra, Tampa, FL (US)

(73) Assignee: University of South Florida (A Florida Non-Profit Corporation), Tampa, FL (US)

(21) Appl. No.: 14/026,165

(22) Filed: Sep. 13, 2013

Related U.S. Application Data
(60) Provisional application No. 61/700,600, filed on Sep. 13, 2012.

(51) Int. Cl. A61K 31/403

(52) U.S. Cl. CPC A61K 31/403
USPC 514/411

(57) ABSTRACT

Embodiments of the present disclosure provide methods of treating a traumatic brain injury with an agent. In particular, embodiments of the present disclosure provide for methods of treating a traumatic brain injury using an agent such as (±)-[3-(9H-carbazol-4-yloxy)-2-hydroxypropyl][2-(2-methoxyphenoxy)ethyl]amine or an isomer, a tautomer, or a prodrug thereof, or pharmaceutically acceptable salt each of these.

### CROSS-REFERENCE TO RELATED APPLICATION

This application claims priority to U.S. provisional application entitled “Methods of treating traumatic brain injury,” having Ser. No. 61/700,600, filed on Sep. 13, 2012, which is entirely incorporated herein by reference.

### BACKGROUND

Traumatic brain injury may be caused by blunt injury such as motor vehicle accidents, falling, penetrating injury, or blast injury. Immediate neuronal, axonal and vascular destruction results from the impact of a blunt, bullet or blast injury. The amount of immediate tissue damage is highly variable depending upon the energy transfer at the point of impact and the medical status of the victim. In the civilian population, at least 200,000-300,000 significant blunt. Methods of reducing brain damage are needed.

### SUMMARY

Embodiments of the present disclosure provide methods of treating a traumatic brain injury with an agent.

An embodiment of the present disclosure includes a method of treating a human having suffered a traumatic brain injury, where the method includes administering to the human a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine, wherein administering includes a dose within 6 hours of the traumatic brain injury.

An embodiment of the present disclosure includes a method of treating a human having suffered a traumatic brain injury, where the method includes administering to the human a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine.

An embodiment of the present disclosure includes a method of treating a patient having suffered a traumatic brain injury, where the method includes administering to the patient a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine or an isomer, a tautomer, or a prodrug thereof, or pharmaceutically acceptable salt each of these.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates that Carvedilol treatment reduces hippocampal damage following tramatic brain injury.

### DISCUSSION

Before the present disclosure is described in greater detail, it is to be understood that this disclosure is not limited to particular embodiments described, and that the various embodiments of the invention may, of course, vary. It is also to be understood that the terminology used herein is for the purpose of describing particular embodiments only, and is not intended to be limiting, because the scope of the present disclosure is to be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, biochemistry, biology, molecular biology, recombinant DNA techniques, pharmacology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes both a single compound and a plurality of compounds. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

As used herein, the terms “treatment”, “treating”, and “treat” are defined as acting upon a traumatic brain injury with an agent to reduce or ameliorate the pharmacologic and/or physiologic effects of the traumatic brain injury and/or its symptoms. “Treatment,” as used herein, covers any treatment of a disease in a patient (e.g., a mammal, typically a human or non-human animal of veterinary interest), and includes: (a) reducing the impact of the traumatic brain injury, (b) impeding progression of the damage caused by the traumatic brain injury, and (c) relieving the traumatic brain injury, i.e., causing regression of the traumatic brain injury and/or relieving one or more traumatic brain injury symptoms.

As used herein, the terms “prophylactically treat” or “prophylactically treating” refers administering an agent prior to the traumatic brain injury.

As used herein, the term “subject” or “patient” includes humans and mammals (e.g., mice, rats, pigs, cats, dogs, and horses). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats.

The term “unit dosage form,” as used herein, refers to physically discrete units suitable as unitary dosages for human and animal subjects, each unit containing a predetermined quantity of an agent calculated in an amount sufficient to produce the desired effect in association with a pharmaceutically acceptable diluent, carrier or vehicle. The specifications for unit dosage forms depend on the particular agent employed and the effect to be achieved, and the pharmacodynamics associated with each compound in the subject.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more such excipients, diluents, carriers, and adjuvants.

As used herein, a “pharmaceutical composition” is meant to encompass an agent suitable for administration to a subject, such as a mammal, especially a human. In general a “pharmaceutical composition” is sterile, and preferably free of contaminants that are capable of eliciting an undesirable response within the subject (e.g., the agent in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, inhalation, and the like.

The term “therapeutically effective amount” as used herein refers to that amount of an agent being administered that will relieve to some extent one or more of the symptoms of the condition of the traumatic brain injury.

“Pharmaceutically acceptable salt” refers to those salts of the agent that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like as well as hydrates.

In the event that embodiments of the disclosed agents form salts, these salts are within the scope of the present disclosure. Reference to an agent of any of the formulas herein is understood to include reference to salts thereof, unless otherwise indicated. The term “salt(s)”, as employed herein, denotes acidic and/or basic salts formed with inorganic and/or organic acids and bases. In addition, when an agent contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an agent may be formed, for example, by reacting the agent with an amount of acid or base, such as an equivalent amount, in a medium such as one in which the salt precipitates or in an aqueous medium followed by lyophilization.

Embodiments of agent that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the agent that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the agents described in the instant disclosure are also contemplated herein.

To the extent that the disclosed active agents (compounds, and salts thereof), may exist in their tautomeric form, all such tautomeric forms are contemplated herein as part of the present disclosure.

All stereoisomers of the agents, such as those that may exist due to asymmetric carbons on the various substituents, including enantiomeric forms (which may exist even in the absence of asymmetric carbons) and diastereomeric forms, are contemplated within the scope of this disclosure. Individual stereoisomers of a compound described herein may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

The term “prodrug” refers to an inactive precursor of an agent that is converted into a biologically active form in vivo. Prodrugs are often useful because, in some situations, they may be easier to administer than the parent compound. They may, for instance, be bioavailable by oral administration whereas the parent compound is not or is less so. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet, 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “administration” refers to introducing an agent into a subject. Preferred routes of administration of the agent include oral administration and intravenous administration. However, any route of administration, such as intravenous, topical, subcutaneous, peritoneal, intraarterial, inhalation, vaginal, rectal, nasal, introduction into the cerebrospinal fluid, or instillation into body compartments, can be used.

### Discussion

Embodiments of the present disclosure provide methods of treating a traumatic brain injury with an agent. In particular, embodiments of the present disclosure provide for methods of treating a traumatic brain injury using an agent such as (±)-[3-(9H-carbazol-4-yloxy)-2-hydroxypropyl][2-(2-methoxyphenoxy)ethyl]amine (also known as Carvedilol) or an isomer, a tautomer, or a prodrug thereof, or pharmaceutically acceptable salt each of these.

Embodiments of the present disclosure provide compositions (including pharmaceutical compositions) including an agent that can be used to treat a subject (e.g., human patient) after having had a traumatic brain injury. In an embodiment, the treatment can include one or more doses of the agent within about 1, 2, 3, 4, 5, 6, 7, 8, 9, 10, 11, 12, 14, 16, 18, 20, 22, or 24 hour(s) of the traumatic brain injury, or a combination thereof. For example, the agent (e.g., the same or different dose) can be administered once or in intervals of about 30 min, 1, 2, 3, 4, 5, 6, 8, 12, or 24 hour(s), as needed for treatment. The dose amount, frequency, and the number of doses, can depend upon the nature of the traumatic brain injury (e.g., cause, severity, extent of injury, damage after the injury, and the like), the subject (e.g., age, health, sex, and the like), the time that the first dose is administered, as well as other medications taken before or after the traumatic brain injury.

The pharse “traumatic brain injury” (also referred to as “an intracranial injury”) can be caused by an impact, strick, force, shock wave (e.g., from an explosion) and the like on the head and/or a sudden acceleration or deceleration so that brain impacts the skull. The traumatic brain injury includes not only the direct damage but also the damage caused by pressure within the skull, changes in the blood flow, and the like. The traumatic brain injury is an acquired brain injury or head injury. The injury can be focal or diffuse. The injury can be mild, moderate, or severe based on variables such as duration of loss of consciousness, Glassgow Coma Score, and/or post traumatic stress amnesia, or the like. The injury can be chronic or acute. The traumatic brain injury can be the result from a traffic accident, crashing or falling, sports activities, result of explosions, military activities, combinations thereof, and the like.

As mentioned above, the agent can include (±)-[3-(9H-carbazol-4-yloxy)-2-hydroxypropyl][2-(2-methoxyphenoxy)ethyl]amine (also known as Carvedilol), derivatives thereof, or an isomer, a tautomer, or a prodrug thereof, or pharmaceutically acceptable salt each of these. The agent, derivatives thereof, or an isomer, a tautomer, or a prodrug thereof, or pharmaceutically acceptable salt each, and methods of making these may be further described in one or more of the following: 4,503,067, 5,071,868, 6,699,997, 7,056,942, 7,268,156, 7,485,663, 7,626,041, 7,649,010, and 7,750,036; each of which is incorporated herein by reference.

Embodiments of the agent can be contained in compositions, pharmaceutical compositions, liquid compositions, gel compositions, and the like, including without limitation controlled release and sustained release formulations.

In an embodiment, a simple and convenient dosing regimen for treating patients having a traumatic brain injury includes any regimen previously shown effective for use of the agent for its known uses. In one embodiment, a BID dosing regimen is employed. In one embodiment, the regimen is 100 mg po BID. Embodiments of the disclosure provides novel unit dose forms of clemizole, including 25 mg, 50 mg, 75 mg, 100 mg, 125 mg, 150 mg, and 200 mg unit dose forms. In one embodiment, the unit dose form is a tablet; in another, the unit dose form is a capsule. In one embodiment, sustained release formulations of agent are in the tablet and capsule form. In another embodiment, the unit dose form is a liquid.

### Pharmaceutical Formulations and Routes of Administration

Embodiments of the present disclosure include an agent formulated with one or more pharmaceutically acceptable excipients, diluents, carriers and/or adjuvants. In addition, embodiments of the present disclosure include the agent formulated with one or more pharmaceutically acceptable auxiliary substances. In particular, one or more agents can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment, the agent is administered to the subject using a convenient means capable of resulting in the desired effect. Thus, embodiments of the present disclosure provide an agent incorporated into a variety of formulations for therapeutic administration. For example, embodiments provide the agent formulated into pharmaceutical compositions by combination with appropriate, pharmaceutically acceptable carriers or diluents, and formulated into preparations in solid, semi-solid, liquid or gaseous forms, such as tablets, capsules, powders, granules, ointments, solutions, suppositories, injections, inhalants and aerosols.

In pharmaceutical dosage forms, the agent may be present in the form of its pharmaceutically acceptable salts, or a subject active agent may be used alone or in appropriate association, as well as in combination, with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent can be utilized in aerosol formulation to be administered via inhalation. Embodiments of the agent can be formulated into pressurized acceptable propellants such as dichlorodifluoromethane, propane, nitrogen and the like.

Furthermore, embodiments of the agent can be made into suppositories by mixing with a variety of bases such as emulsifying bases or water-soluble bases. Embodiments of the agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more agents. Similarly, unit dosage forms for injection or intravenous administration may comprise the agent in a composition as a solution in sterile water, normal saline or another pharmaceutically acceptable carrier.

Embodiments of the agent can be formulated in an injectable composition in accordance with the present disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

In an embodiment, the agent is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent can be accomplished using any of a variety of refillable, pump systems. Pumps provide consistent, controlled release over time. In some embodiments, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent is delivered using an implantable drug delivery system, e.g., a system that is programmable to provide administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the iagent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Compositions of the present disclosure may be used with a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Exemplary biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) is provided in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target, i.e., the brain, thus requiring only a fraction of the systemic dose. Other controlled release systems are discussed in the review by Langer (1990). Science 249:1527-1533.

In another embodiment, the compositions of the present disclosure (as well as combination compositions, whether administered as separate unit dose forms or together in a single dose form) may be impregnated into absorptive materials, such as sutures, bandages, and gauze, or coated onto the surface of solid phase materials. Other delivery systems of this type will be readily apparent to those skilled in the art.

### Dosages

Embodiments of the agent can be administered to a subject in one or more doses.

In an embodiment, the agent can be administered in an amount of about 10 mg to 1000 mg per dose, e.g., about 10 mg to 20 mg, about 20 mg to 25 mg, about 25 mg to 50 mg, about 50 mg to 75 mg, about 75 mg to 100 mg, about 100 mg to 125 mg, about 125 mg to 150 mg, about 150 mg to 175 mg, about 175 mg to 200 mg, about 200 mg to 225 mg, about 225 mg to 250 mg, about 250 mg to 300 mg, about 300 mg to 350 mg, about 350 mg to 400 mg, about 400 mg to 450 mg, about 450 mg to 500 mg, about 500 mg to 750 mg, or about 750 mg to 1000 mg per dose.

In an embodiment, the amount of the agent per dose is determined on a per body weight basis. For example, in an embodiment, the agent can be administered in an amount of about 0.5 mg/kg to 100 mg/kg, e.g., about 0.5 mg/kg to 1 mg/kg, about 1 mg/kg to 2 mg/kg, about 2 mg/kg to 3 mg/kg, about 3 mg/kg to 5 mg/kg, about 5 mg/kg to 7 mg/kg, about 7 mg/kg to about 10 mg/kg, about 10 mg/kg to 15 mg/kg, about 15 mg/kg to 20 mg/kg, about 20 mg/kg to 25 mg/kg, about 25 mg/kg to 30 mg/kg, about 30 mg/kg to 40 mg/kg, about 40 mg/kg to 50 mg/kg per dose, about 50 mg/kg to 60 mg/kg, about 60 mg/kg to 70 mg/kg, about 70 mg/kg to 80 mg/kg, about 80 mg/kg to 90 mg/kg, or about 90 mg/kg to 100 mg/kg, or more than about 100 mg/kg.

Those of skill will readily appreciate that dose levels can vary as a function of the specific agent, the severity of the symptoms and the susceptibility of the subject to side effects. Preferred dosages for a given compound are readily determinable by those of skill in the art by a variety of means.

In an embodiment, multiple doses of the agent are administered. The frequency of administration of the agent can vary depending on any of a variety of factors, e.g., severity of the symptoms, and the like.

The duration of administration of the agent, e.g., the period of time over which the agent is administered, can vary, depending on any of a variety of factors, e.g., patient response, etc. For example, the gent can be administered over a period of time of about 1 hour to a few hours, to 12 hours to one day to one week or more.

### Routes of Administration

Embodiments of the present disclosure provide methods for treating a traumatic brain injury comprising the administration of the agent to a subject (e.g., a human) using any available method and route suitable for drug delivery.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An agent can be administered in a single dose or in multiple doses. The agent described herein is suitable for oral administration, which may be preferable for most patients.

Embodiments of the agent can be administered to a host using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not limited to, oral and rectal (e.g., using a suppository) delivery.

Methods of administration of the agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

### EXAMPLES

The following examples are put forth so as to provide those of ordinary skill in the art with a complete disclosure and description of how to make and use the present disclosure, and are not intended to limit the scope of the disclosure nor are they intended to represent that the experiments below are all or the only experiments performed.

### Example 1

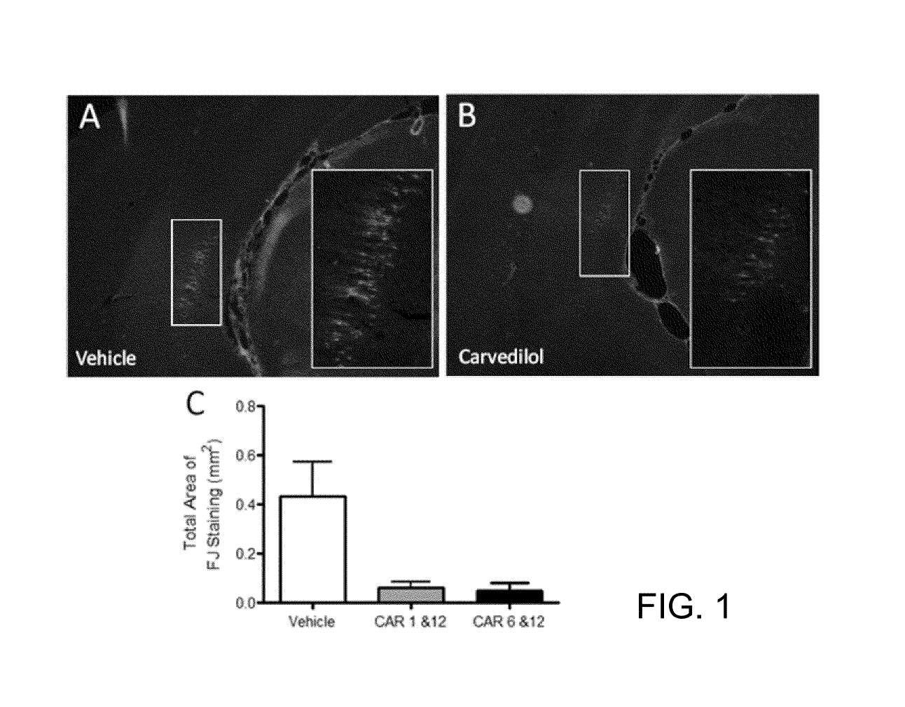

FIG. 1 illustrates that Carvedilol treatment reduces hippocampal damage following TBI. Representative images of the CA3 layer of the hippocampus in rats 24 hrs following TBI, vehicle treated (A) and carvedilol treated (B) at 1 and 12 hrs following the insult. Carvedilol administration at 1 and 12 hrs or at 6 and 12 significantly reduces hippocampal damage following TBI (C) p<0.0293 using ANOVA with a Dunnett's post-hoc test. Images were taken at 4× magnification. Dosing was subcutaneous and the vehicle is 2.5% DMSO in saline.

### Methods

### Animals

All animal procedures were conducted in accordance with the NIH Guide for the Care and Use of Laboratory Animals following a protocol approved by the Institutional Animal Care and Use Committee at the University of South Florida. Male Sprague-Dawley rats (Harlan, Indianapolis, Ind.) weighing 250 to 300 g were housed in a climate-controlled room with water and laboratory chow available ad libitum. A total of 33 animals were used in this study.

Induction of Lateral Fluid Percussion Injury (LFPI) Animals were anesthetized using a mixture of ketamine (90 mg/kg)/xylazine (10 mg/kg) (IP). To deliver LFPI, a 1 mm diameter craniotomy was performed centered at 2 mm lateral and 2.3 mm caudal to the bregma on the right side of the midline. A female luer-lock hub was implanted at the craniotomy site and secured with dental cement. The FPI device was then fastened to the luer-lock. All tubing was checked to ensure that no air bubbles had been introduced, after which a mild impact ranging from 2.0-2.2 atm. was administered. Impact pressures were measured using a transducer attached to the point of impact on the fluid percussive device. The luer-lock was then detached, the craniotomy hole was sealed with bone wax and the scalp was sutured. Ketoprofen (5 mg/kg) was administered to minimize postsurgical pain and discomfort. Rats were then replaced in their home cages and allowed to recover. Animals were excluded from further tests if the impact did not register between 2.0 and 2.2 atm. or if the dura was disturbed during the craniotomy prior to impact. In sham (control) animals, craniotomy was performed at the same coordinates as the TBI animals but no impact was delivered.

### Tissue Collection

Animals were deeply anesthetized with ketamine (75 mg/kg) and xylazine (7.5 mg/kg) 24 or 48 hours after TBI. Thymuses and spleens were removed and immediately snap frozen on dry ice. Animals were then perfused with 0.9% saline followed by 4% paraformaldehyde in phosphate buffer (pH 7.4). The brains were harvested, post-fixed in 2% paraformaldehyde and saturated with increasing sucrose concentrations (20% to 30%) in phosphate-buffered saline (PBS, pH 7.4). Brains were then frozen, sectioned coronally at 30 μm thickness using a cryostat, thaw-mounted onto glass slides and stored at −20° C. prior to staining

### Fluoro-Jade Histochemistry

Fluoro-Jade (Histochem, Jefferson, Ark.) staining was performed to label degenerating neurons. Thaw-mounted sections were placed in 100% ethanol for 3 minutes followed by 70% ethanol and deionized water for 1 minute each. Sections were then oxidized using a 0.06% KMnO₄ solution for 15 minutes followed by three rinses in ddH2O for 1 minute each. Sections were then stained in a 0.001% solution of Fluoro-Jade in 0.1% acetic acid for 30 min. Slides were rinsed, dried at 45° C. for 20 min, cleared with xylene, and cover-slipped using DPX mounting medium (Electron Microscopy Sciences, Ft. Washington, Pa.).

### Image Analysis and Quantitation

All quantitation was performed using the NIH Image J software. For immunohistochemical analysis, images were acquired using an Olympus IX71 microscope controlled by DP70 manager software (Olympus America Inc., Melville, N.Y.). Photomicrographs captured at 200× magnification with an Olympus DP70 camera were used for quantification. Images were taken at the same exposure and digital gain settings for a given magnification to minimize differential background intensity or false-positive immunoreactivity across sections. The channels of the RGB images were split and the green channel was used for quantitation of the FJ staining images. The single channel images were then adjusted for brightness and contrast to exclude noise pixels. The images were also adjusted for the threshold to highlight all the positive cells to be counted and a binary version of the image was created with pixel intensities 0 and 255. Particle size was adjusted to exclude the small noise pixels from the count. Circularity was adjusted to between 0 and 1 to discard any cell fragments, processes or tissue aggregates resulting in false labeling from the quantitation. The same specifications were used for all sections. Cell counts of sections from 3.5, 4.5 and 5.5 mm caudal to the bregma were summed to represent the number of positive cells from each brain. The results for the FJ were expressed as mean number of positive cells±S.E.M.

### Statistical Analysis

All data are presented as mean±S.E.M. Statistical significance was evaluated by one-way ANOVA with Bonferroni's post-hoc test. A p value of less than 0.05 was considered statistically significant for all comparisons.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to the measuring technique and the numerical value. In addition, the phrase “about ‘x’ to ‘y’” includes “about ‘x’ to about ‘y’”.

While only a few embodiments of the present disclosure have been shown and described herein, it will become apparent to those skilled in the art that various modifications and changes can be made in the present disclosure without departing from the spirit and scope of the present disclosure. All such modification and changes coming within the scope of the appended claims are intended to be carried out thereby

What is claimed is:

1. A method of treating a human having suffered a traumatic brain injury, the method comprising administering to the human a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine, wherein administering includes a dose within 6 hours of the traumatic brain injury.

2. The method of claim 1, wherein administering includes multiple doses after the traumatic brain injury.

3. The method of claim 1, wherein administering a second dose at about 8 to 12 hours after the traumatic brain injury.

4. The method of claim 1, wherein administering occurs within 1 hour of the traumatic brain injury.

5. A method of treating a human having suffered a traumatic brain injury, the method comprising administering to the human a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine.

6. The method of claim 5, wherein administering occurs within 1 hour of the traumatic brain injury.

7. The method of claim 5, wherein administering occurs at about 1 to 6 hours after the traumatic brain injury.

8. The method of claim 5, wherein administering occurs at about 6 to 12 hours after the traumatic brain injury.

9. A method of treating a patient having suffered a traumatic brain injury, the method comprising administering to the patient a therapeutically effective amount of an agent to treat the traumatic brain injury, wherein the agent is (±)-[3-(9H-carbazol-4-yloxy)-2-hydroxypropyl][2-(2-methoxyphenoxy)ethyl]amine or an isomer, a tautomer, or a prodrug thereof, or pharmaceutically acceptable salt each of these.

10. The method of claim 9, wherein administering occurs within 1 hour of the traumatic brain injury.

11. The method of claim 9, wherein administering occurs within 6 hours of the traumatic brain injury.

12. The method of claim 9, wherein administering occurs at about 1 to 6 hours after the traumatic brain injury.

13. The method of claim 9, wherein administering occurs at about 6 to 12 hours after the traumatic brain injury.

14. The method of claim 9, wherein administering includes multiple doses after the traumatic brain injury.

15. The method of claim 14, wherein administering includes a first dose within 6 hours of the traumatic brain injury and a second dose at about 8 to 12 hours after the traumatic brain injury.
